(19) United States
(12) Patent Application Publication
(10) Pub. No.: US 2019/0358413 A1
(43) Pub. Date: Nov. 28, 2019

(54) DEVICE FOR REDUCTION OF PERCEIVED PAIN IN PATIENTS

(71) Applicant: Lane Biomedical, LLC, Columbus, GA (US)

(72) Inventor: Joshua Edward Lane, Columbus, GA (US)

(21) Appl. No.: 16/524,809

(22) Filed: Jul. 29, 2019

Related U.S. Application Data
(63) Continuation-in-part of application No. 14/752,310, filed on Jun. 26, 2015.
(60) Provisional application No. 62/031,576, filed on Jul. 31, 2014.

(51) Int. Cl. A61M 5/42; A61F 7/02

(52) U.S. Cl. CPC A61M 5/422; A61F 2007/0052; A61F 2007/0219; A61F 7/02

(57) ABSTRACT

Various embodiments of the present invention are directed to a device for the reduction perceived pain in patients. Various embodiments comprise a flexible shell comprising a pliable material and a first non-planar surface and a second surface opposite the first surface. The flexible shell defines an interior cavity located intermediate the first surface and the second surface and filled at least in part by a first substance. The first surface comprises a plurality of protrusions distributed therein and configured to be depressed into the skin of the patient. The pain reducing device may execute a cooling functionality upon being moved from a first position to a second position. When cooled and depressed against the patient's skin, the pain reducing device provides a sensory confusion sensation to the patient, thereby reducing perceived pain caused by a needle insertion.

### CROSS-REFERENCE TO RELATED APPLICATIONS

This patent application is a continuation-in-part of U.S. application Ser. No. 14/752,310, filed on Jun. 26, 2015, and entitled “Device for Reduction of Needle Insertion Site Pain in Patients”, which claims priority to Provisional Application Ser. No. 62/031,576 entitled “Device for Reduction of Needle Insertion Site Pain in Patients,” filed Jul. 31, 2014, the contents of both of which are incorporated herein by reference in its entirety.

### BACKGROUND

In an effort to reduce the perceived pain and the general unpleasantness of needle pricks associated with medical shots and blood testing, devices and methods may be used to distract patients from pain associated with a needle insertion. Such distraction may be accomplished by having the patient focus on an unrelated stimulant (e.g., a conversation with a third-party) or by confusing the nerves around the needle insertion site.

Various devices have been developed for confusing the nerves around a needle insertion site. However, many of these devices require power sources (e.g., batteries) that must be replaced or charged periodically. Other existing devices utilize only a single method of confusing nerves around the pain site, and therefore may not maximize potential pain relief for a patient. Moreover, existing devices are generally configured to confuse nerves on a single side of an insertion site, and therefore these devices may be difficult to align in an appropriate location to maximize pain reduction.

Therefore a need exists for an unpowered device capable of effectively reducing pain caused by needle insertion that may be easily aligned around the needle insertion site to maximize pain reduction associated with needle insertion.

### BRIEF SUMMARY

Various embodiments are directed to a pain reducing device comprising: a flexible shell comprising a first non-planar surface and a second surface opposite the first surface; and an interior cavity defined by the flexible shell and located intermediate the first surface and the second surface of the flexible shell, wherein: the flexible shell comprises a pliable material; the first surface comprises a plurality of protrusions; and the interior cavity is at least partially filled with a first substance. In various embodiments, the first non-planar surface may define a curved profile having a first radius of curvature when the pain reducing device is in a first position, the first position being a natural curved configuration with the first surface unaffected by any externally applied forces. In various embodiments, the first surface may define a curved profile having a second radius of curvature when the pain reducing device is in a second position, wherein the second radius of curvature is larger than the first radius of curvature, and wherein the second position defines a substantially flat configuration of the first surface affected by an applied force. In various embodiments, the first surface may define a curved profile having a third radius of curvature when the pain reducing device is in a third position, wherein the third radius of curvature is larger than the first radius of curvature and smaller than the second radius of curvature, and wherein the third position defines a configuration of the first surface corresponding to a profile of an area surrounding a location of direct contact with a patient. In various embodiments, the second surface may be a nonplanar surface defining a curved profile that corresponds to the curved profile of the first surface in the first position. In various embodiments, the freezable device may further comprise at least one passage extending through the device from the first surface to the second surface, wherein a passage is configured to surround at least a portion of a location of direct contact with a patient. In various embodiments, the flexible shell may comprise a perimeter surface surrounding the shell and extending between the first surface and the second surface to define the interior cavity of the shell; and wherein the passage defines a passage surface at least partially surrounding the passage and extending between the first surface and the second surface; and wherein the perimeter surface comprises the passage surface.

In various embodiments, the passage may define a first hole extending through the first surface and a second hole extending through the second surface, and a passage perimeter surface extending between the first hole and the second hole. In various embodiments, the pain reducing device may comprise two or more passages extending through the device from the first surface to the second surface. In various embodiments, the flexible shell may be sterile. In various embodiments, the first substance may be a gel. In various embodiments, the pain reducing device may further comprise a second substance within the interior cavity of the flexible shell, wherein the interior cavity defines a first portion and a second portion separated by a barrier when the flexible device is in the first position, and wherein the first substance and the second substance are positioned within the first portion and the second portion, respectively. In various embodiments, the barrier may be configured to break when the pain reducing device is moved from a first position to a second position so as to initiate an interaction between the first substance and the second substance, wherein the first substance and the second substance are configured such that the interaction activates a cooling functionality of the pain reducing device. In various embodiments, the first substance may be water and the second substance may be ammonium chloride.

Various embodiments are directed to a method of reducing perceived pain during a needle insertion into a patient, the method comprising for the steps of: providing a pain reducing device comprising a first surface and a second surface opposite the first surface, wherein the first surface comprises a plurality of protrusions configured to be pressed against the patient, and wherein the first surface is a substantially nonplanar surface defining a curved profile having a first radius of curvature when the pain reducing device is in a first position, wherein the first position defines a natural curved configuration of the first surface unaffected by external forces; applying a flattening force to the pain reducing device such that the pain reducing device is reconfigured from the first position to a second position, wherein the first surface defines a curved profile having a second radius of curvature when the pain reducing device is in the second position, wherein the second radius of curvature is larger than the first radius of curvature; positioning the pain reducing device adjacent a patient such that at least a portion of the first surface is in contact with at least a location of direct contact with a patient; after positioning the device adjacent the patient, unapplying the flattening force from the pain reducing device such that the pain reducing device is reconfigured from the second position to a third position, wherein the first surface defines a curved profile having a third radius of curvature when the pain reducing device is in a third position, wherein the third radius of curvature is larger than the first radius of curvature and smaller than the second radius of curvature, and wherein the third position defines a configuration of the first surface corresponding to a profile of an area surrounding a location of direct contact with a patient; applying a pressing force to the second surface such that the plurality of protrusions is pressed against the patient's skin.

In various embodiments, the second position may define a substantially flat configuration of the first surface affected by the flattening force. In various embodiments the method may further comprise activating a cooling functionality of the pain reducing device. In various embodiments, activating a cooling functionality of the pain reducing device may comprise breaking a barrier within an interior cavity of the pain reducing device so as to initiate an interaction between a first substance and a second substance positioned within the interior cavity, wherein the first substance and the second substance are separated by the barrier when the pain reducing device is in the first position. In various embodiments, the method may further comprise inserting a needle into the patient's skin, wherein inserting the needle into the patient's skin comprises inserting the needle through a passage extending through the pain reducing device between the first surface and the second surface and into the patient's skin. In various embodiments, the needle may be inserted into the patient's skin proximate the location of direct contact with a patient. In various embodiments, a membrane may cover at least one end of the passage along a perimeter surface of the pain reducing device, and wherein inserting the needle through the passage and into the patient's skin further comprises puncturing the membrane. In various embodiments, the method may further comprise sterilizing the pain reducing device prior to pressing the pain reducing device against the patient's skin.

### BRIEF DESCRIPTION OF THE DRAWINGS

Reference will now be made to the accompanying drawings, which are not necessarily drawn to scale, and wherein:

FIG. 1 is a top-perspective view of a freezable device according to various embodiments of the present invention;

FIG. 2 is a side perspective view of a freezable device according to various embodiments of the present invention;

FIG. 3 is a side view of a freezable device according to various embodiments of the present invention;

FIG. 4 is a side view of a freezable device according to various embodiments of the present invention;

FIG. 5 is a bottom view of a freezable device according to various embodiments of the present invention;

FIGS. 6A-6C are bottom views of freezable devices according to various embodiments of the present invention; and

FIG. 7 is a bottom view of a freezable device in use according to various embodiments of the present invention.

FIG. 8 illustrates a flow diagram of an exemplary method of reducing perceived pain during a needle insertion into a patient.

FIG. 9 is a perspective view of a freezable device in use according to various embodiments of the present invention.

### DETAILED DESCRIPTION

The present invention will now be described more fully hereinafter with reference to the accompanying figures, in which some, but not all embodiments of the invention are shown. Indeed, the invention may be embodied in many different forms and should not be construed as limited to the embodiments set forth herein. Rather, these embodiments are provided so that this disclosure will satisfy applicable legal requirements.

### Overview

A device for reducing perceived pain associated with a needle insertion by confusing nerves surrounding a needle insertion site is provided. In various embodiments, the device (hereinafter the “freezable device”) may comprise a freezable pain reducing device, but should not be understood to be limited to configurations in which the device is freezable; in various embodiments, the freezable device may not be freezable. In various embodiments, the device may comprise a freezable device having one or more passages extending therethrough and having a plurality of raised protrusions on an exterior surface of the freezable device. In various embodiments, the freezable device may comprise a pliable exterior shell having the plurality of raised protrusions on the exterior surface of the shell. However, in various embodiments the freezable device may comprise a rigid and/or semi-rigid exterior shell. The exterior shell may define an interior cavity filled at least partially with a freezable substance (e.g., a freezable liquid and/or a freezable gel). Alternatively, the interior cavity may be filled with a gel configured to maintain a gelatinous state at low temperatures. As a non-limiting example, the gel may be configured to maintain a gelatinous state at 0 degrees Fahrenheit, such that the freezable device may maintain flexibility when subjected to low temperatures. As yet another alternative, the interior cavity may be filled with a plurality of small solid and/or gelatinous objects (e.g., pellets) such that the freezable device maintains flexibility at low temperatures. In various embodiments, the freezable device may be sufficiently flexible to be used in anatomic areas with variable topography (e.g., near a patient's nose) at low temperatures.

In various embodiments, the freezable device may be sterilized prior to use. Specifically, a plurality of sterile freezable devices may be stored within a dispenser in a freezer and may be removed as needed. In various embodiments, the freezable device may be configured to be used for a single needle insertion prior to disposal.

Various embodiments provide a method of using a freezable device for reducing perceived pain associated with a needle insertion. In various embodiments, the method may comprise steps for freezing a freezable device having an exterior surface including a plurality of protrusions, one or more passages extending through the freezable device, and an interior cavity filled with a gel or freezable liquid. After chilling the freezable device, an insert may be placed within at least one of the one or more passages extending through the thickness of the freezable device. Following placement of the insert, the freezable device may be placed over a needle insertion site such that a needle may be inserted through the passage and into a patient's skin at the needle insertion site. The freezable device may be pressed firmly into the patient's skin prior to insertion of the needle. The needle may then be inserted through the passage and into the patient's skin. In various embodiments, a user may proceed with placement of the freezable device and insertion of the needle without placing an insert within one of the one or more passages.

### Freezable Device

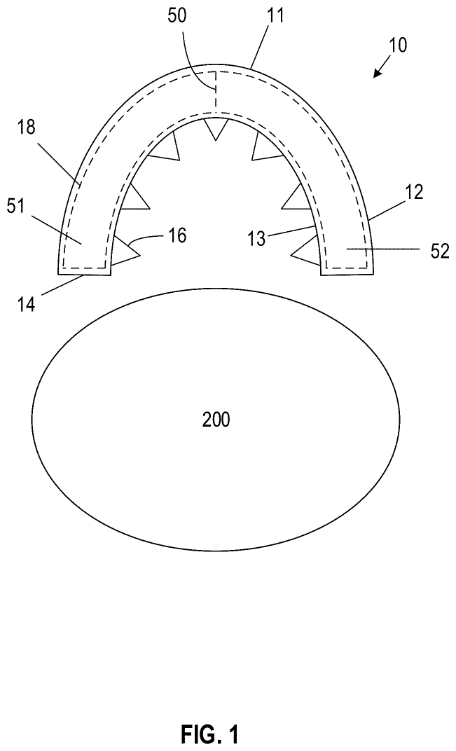

FIG. 1 is a side view of a freezable device 10 according to various embodiments of the present invention. As illustrated in FIG. 1, the freezable pain reducing device 10 comprises a flexible shell 11 defining an interior cavity 18. In various embodiments, the shell 11 comprises a pliable material (e.g., a plastic material, rubber material, and/or the like), such that the freezable device 10 may be configured in each of a first position, a second position, and a third position, as described herein. Moreover, the pliable material may be configured to maintain flexibility at the low temperatures typical of a residential, commercial, or medical freezer (e.g., temperatures between −30 degrees and 10 degrees Fahrenheit, and more preferably temperatures between −15 degrees and 0 degrees Fahrenheit). The shell 11 may comprise a first nonplanar surface 12 and a second surface 13 opposite the first surface. The shell 11 may additionally define a perimeter surface 14 extended around the perimeter of the shell and extending between the first surface 12 and the second surface 13. The interior cavity 18 may be defined by the flexible shell 11 and located intermediate the first non-planar surface 12 and the second surface 13.

As illustrated in FIG. 1, the freezable device 10 may be configured to be arranged in a first position, wherein the first position may be the natural curved configuration of the flexible shell 11 in which the first surface 12 is unaffected by any externally applied forces. In various embodiments, the first non-planar surface 12 may define a curved profile having a first radius of curvature when the freezable device 10 is in the first position. In various embodiments, the second surface 13 may be a nonplanar surface defining a curved profile that corresponds to the curved profile of the first surface 12.

Moreover, in various embodiments, the interior cavity 18 may be filled at least in part with a first substance. In various embodiments, the first substance may comprise a freezable substance (e.g., a freezable liquid and/or a freezable gel). For example, the interior cavity 18 may be filled at least in part with water. Alternatively, the interior cavity 18 may be filled with a gel configured to maintain a gelatinous state at low temperatures. As a non-limiting example, the gel may be configured to maintain a gelatinous state at 0 degrees Fahrenheit, such that the freezable device 10 may maintain flexibility when subjected to low temperatures. As yet another alternative, the interior cavity may be filled with a plurality of small solid and/or gelatinous objects (e.g., pellets) such that the freezable device 10 maintains flexibility at low temperatures.

As illustrated in FIG. 1, the freezable device 10 may further comprise a second substance within the interior cavity 18 of the flexible shell 11. In such a configuration, when the flexible shell is in the first position, the interior cavity 18 defines a first portion 51 and a second portion 52 separated by a barrier 50. In various embodiments, the first substance and the second substance may be positioned within the first portion 51 and the second portion 52, respectively. In various embodiments, the barrier may be configured to break when the freezable device 10 is moved from a first position to a second position, so as to initiate an interaction between the first substance and the second substance. In various embodiments, the first substance and the second substance may be configured such that the interaction of the two substances may activate a cooling functionality of the freezable device 10, wherein the cooling functionality provides a cooling sensation to a patient when the patient body 200 is engaged with the freezable device 10. In various embodiments, for example, the first substance may be water and the second substance may be ammonium chloride.

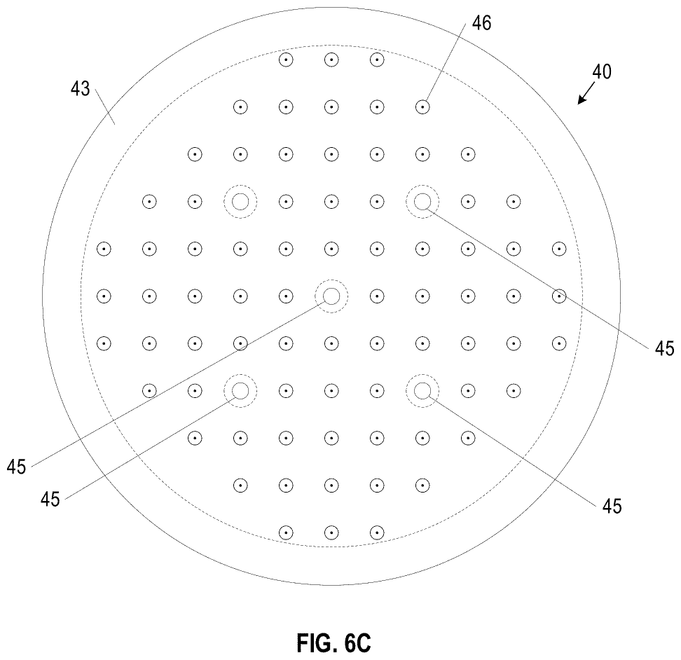

In various embodiments, the shell 11 may comprise a rigid and/or semi-rigid material (e.g., a hard plastic, metal, and/or the like). As shown in FIG. 1, the shell 11 may have a circular shape, although the shell 11 may have any of a variety of other shapes, such as a square shape, a triangular shape, and/or the like (as discussed in reference to FIGS. 6A and 6B herein). The shell 11 may have any of a variety of sizes. As non-limiting examples the shell 11 may have a 1-inch outer diameter or a 2-inch outer diameter.

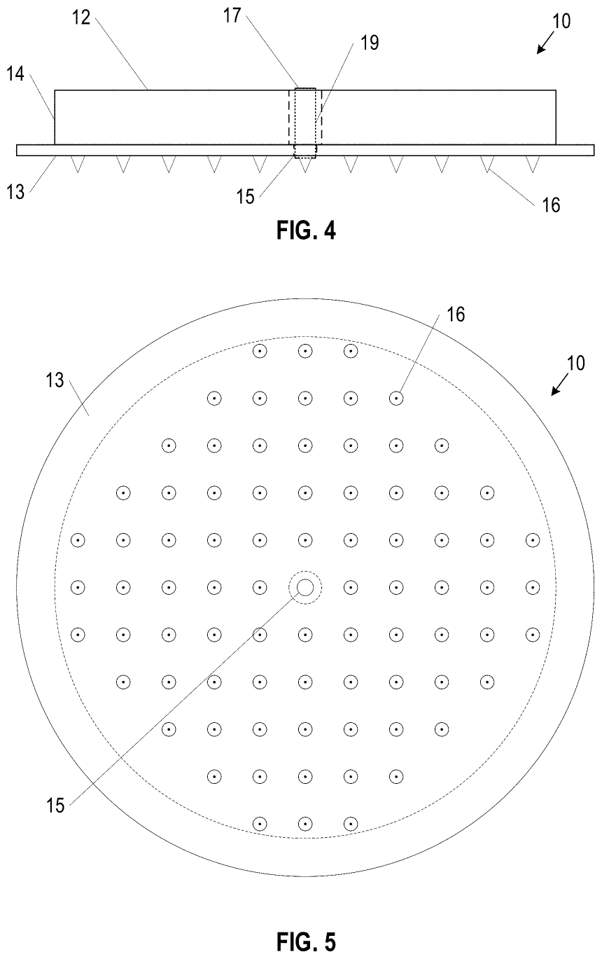

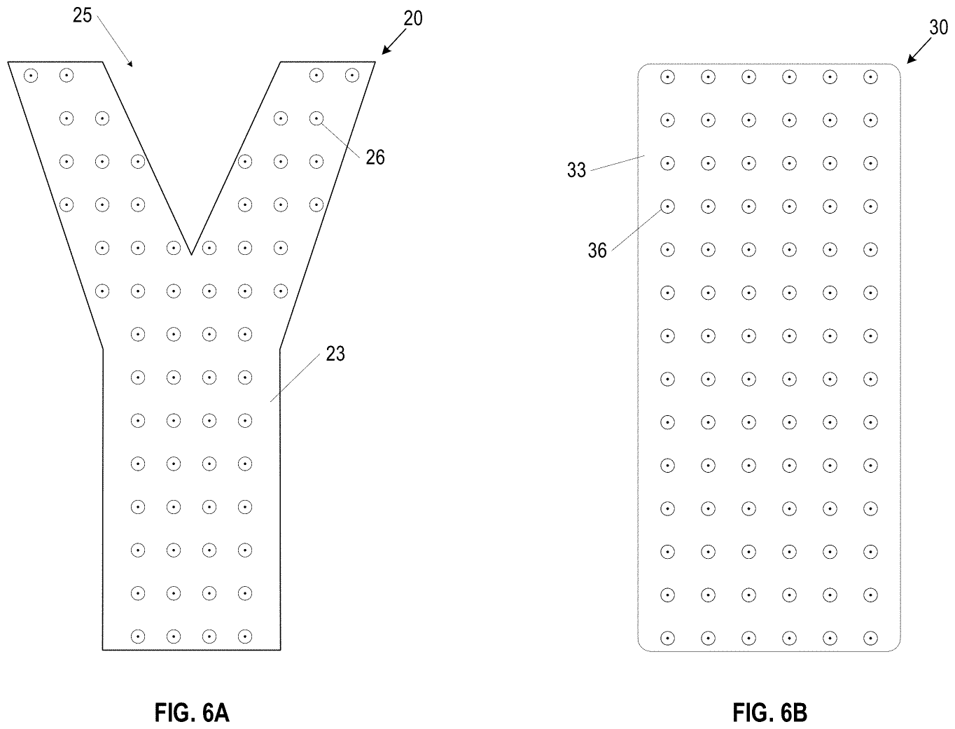

Various embodiments additionally comprise a plurality of protrusions on at least one of the first surface 12 and the second surface 13. As shown in FIGS. 4 and 5, which are schematic side- and bottom-views of a freezable device 10, respectively, the plurality of protrusions 16 may be distributed across the second surface 13. In various embodiments, the various protrusions 16 may comprise conical protrusions, although the protrusions may comprise any of a variety of other shapes (e.g., pyramid shaped, frustum shaped, trapezoidal, hemispherical, cubical, cylindrical, and/or the like). In various embodiments having conical or pyramid shaped protrusions, each protrusion 16 may have a dull point, such that firmly pressing the plurality of protrusions into a patient's skin will not cause the patient to experience pain. As illustrated in FIGS. 4 and 5, the plurality of protrusions 16 may be spaced at least substantially uniformly across the first surface 12 and/or the second surface 13.

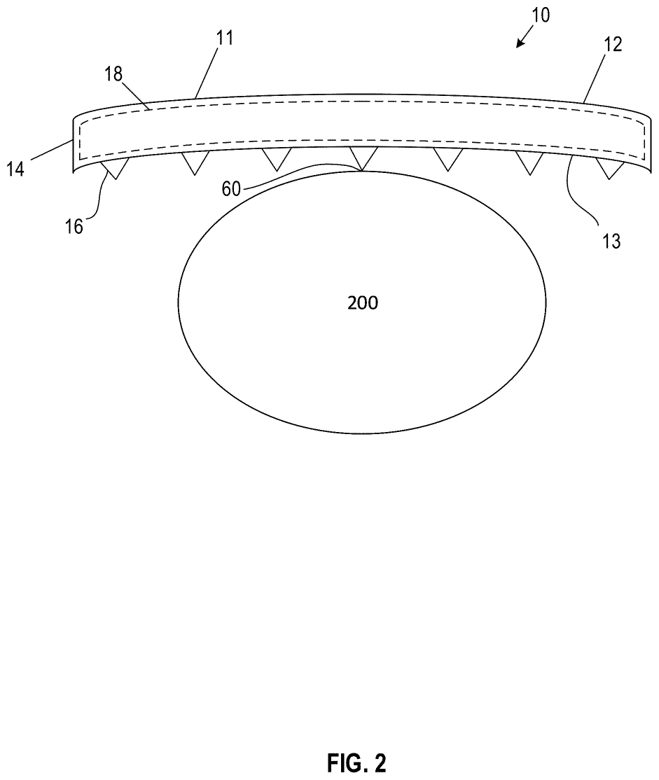

FIG. 2 is a side view of a freezable device 10 according to various embodiments of the present invention. As illustrated in FIG. 2, the freezable device 10 may be configured to be arranged in a second position, wherein the second position may define a substantially flat configuration of the flexible shell 11 in which the first surface 12 is affected by an applied force. In various embodiments, the first non-planar surface 12 may define a substantially flat profile having a second radius of curvature when the freezable device 10 is in the second position. The second radius of curvature may be larger than the first radius of curvature, as described herein. In various embodiments, the force which affects the first surface 12 when the freezable device 10 is in the second position may be a flattening force. As shown in FIG. 2, the freezable device 10 may be configured to engage a patient at least at a portion of a location of direct contact 60.

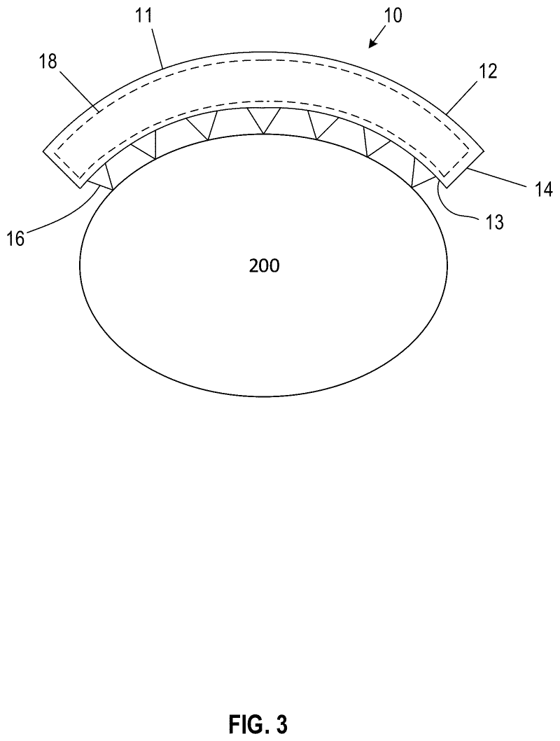

FIG. 3 is a side view of a freezable device 10 according to various embodiments of the present invention. As illustrated in FIG. 3, the freezable device 10 may be configured to be arranged in a third position, wherein the third position may define a curved configuration of the flexible shell 11 in which the first surface 12 is affected by a force received from at least a portion of an area surrounding the location of direct contact 60 with the patient 200. In various embodiments, the first non-planar surface 12 may define a substantially curved profile having a third radius of curvature when the freezable device 10 is in the third position. The third radius of curvature may be larger than the first radius of curvature and smaller than the second radius of curvature, as described herein. In various embodiments, the third position may define a configuration of the first surface 12 corresponding to a profile of an area surrounding a location of direct contact 60 with a patient 200. In various embodiments, the shell 11 comprises a pliable material, such that the freezable device 10 may conform to anatomical features of a patient to reduce needle insertion pain at a plurality of locations on a patient's body. For example, the freezable device 10 may be configured to conform to the bridge of a patient's nose, the area adjacent a patient's eye socket, and/or the like. In various embodiments, the force which affects the first surface 12 when the freezable device 10 is in the third position may be a reciprocal force from the patient body 200 spawned in response to a pressing force being applied to the patient body 200 from the freezable device 10. In various embodiments, as shown in FIG. 3, the freezable device 10 may be configured to apply a pinching force to the patient body 200 via one or more of the protrusions 16 of the plurality of protrusions 46, despite a configuration wherein the freezable device 10 is not being pressed against the patient's body 200 by an external party (i.e. a doctor, a nurse, a patient, etc.). Because the first position of the freezable device 10 represents the natural configuration of the device, and because the first surface 12 of the freezable device 10 has a larger radius of curvature in the third position than in the first position, the pinching force applied to the patient body 200 by the freezable device 10 in the third position is result of the patient body 200 preventing the flexible shell 11 comprised of pliable material from returning to the first position (i.e. its natural configuration). Additionally, the freezable device 10 may be configured to apply a pressing force to the patient body 200 via one or more of the protrusions 16 of the plurality of protrusions 46 being pressed against the patient's body 200 by an external party (i.e. a doctor, a nurse, a patient, etc.). In various embodiments, both a pressing force and a pinching force may be applied to the patient body 200. In various embodiments, a pressing force and a pinching force may be applied to the patient body in different directions relative to the surface of the patient body 200. In various embodiments, for example, the pressing force may be applied to the patient body 200 in a direction perpendicular to the surface of the patient body 200, while the pinching force may be applied to the patient body 200 in a different direction corresponding to the third radius of curvature to the surface of the patient body 200. In such a configuration, the pinching force and the pressing force may respectively provide different sensory confusion sensation to the patient, thereby providing an increased reduction in perceived pain caused by a needle insertion.

As shown in FIG. 4, the freezable device 10 may have a passage 15 extending therethrough, between the first surface 12 and the second surface 13. As shown in FIG. 4, the passage 15 may comprise a hole (e.g., a circular hole) extending through the entirety of the shell 11 such that the hole extends through the first surface 12 and the second surface 13. In various embodiments, the passage 15 may define a substantially cylindrical hole extending through the freezable device 10. In various embodiments, the passage 15 may define a passage surface 19 at least partially surrounding the passage 15 and extending between the first surface 12 and the second surface 13. In various embodiments, the passage 15 may define a first hole extending through the first surface 12 and a second hole extending through the second surface 13, and a passage surface 19 extending between the first hole and the second hole. In various embodiments, the perimeter surface 14 may comprise the passage surface 19. As shown in FIG. 4, the shell 11 may be enclosed such that the interior cavity 18 is sealed, and thus the passage 15 may define a passage surface extending around the perimeter of the passage and between the first surface 12 and the second surface 13. In various embodiments, the passage 15 may be configured to surround at least a portion of a location of direct contact 60 with a patient 200. In various embodiments, the freezable device 10 may comprise two or more passages 15.

In various embodiments, one or more membranes may cover one or both ends of the passage 15, such that the membrane may be punctured by a needle when inserted through the passage 15. As a non-limiting example, the one or more membranes may be defined as a portion of the first surface 12 and/or the second surface 13 extending across the passage 15.

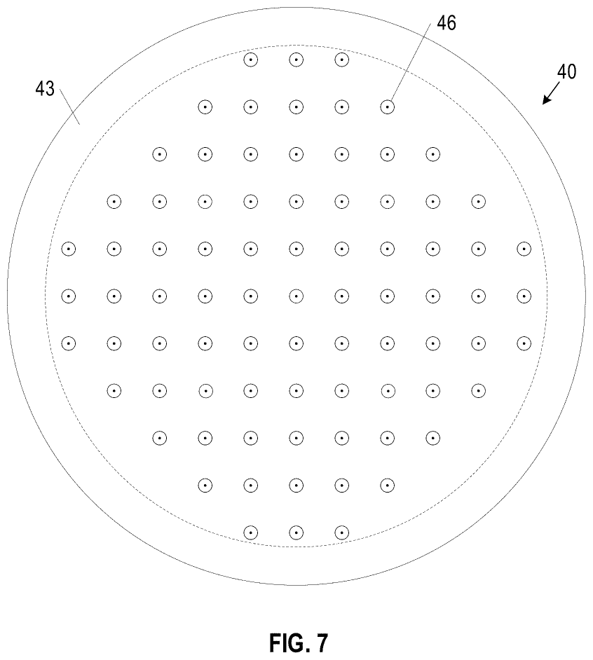

As illustrated in FIGS. 1-7, the freezable device 10 may have a thin profile, such that the thickness of the freezable device 10 is substantially smaller than the length, width, and/or diameter of the freezable device 10. The one or more passages 15 may extend through the freezable device 10 parallel to the thickness, such that the freezable device 10 maintains a large surface area that may be placed in contact with a patient's skin during insertion of a needle through one of the one or more passages 15 into the patient's skin. As shown in FIG. 4, the one or more of the passages 15 may extend through the thickness of the freezable device 10 at the center of the freezable device, although one or more of the passages 15 may extend through the thickness of the freezable device at any location.

Moreover, as shown in FIG. 4, the freezable device 10 may additionally comprise one or more inserts 17 configured to be inserted into one of the one or more passages 15. In various embodiments, the one or more inserts 17 may be provided to a device user in a sterilized state, such that potentially harmful toxins, bacteria, and/or other harmful substances are prevented from contacting a needle prior to insertion into a patient's skin. The one or more inserts 17 may be at least substantially cylindrical in shape, having a hollow center and open ends. In various embodiments, the one or more inserts 17 may have an engagement feature on an exterior surface of the insert 17 configured to engage a corresponding feature on the freezable device 10, such that the insert 17 may remain operatively connected at least partially within the passage 15 of the freezable device 10 during use. The inserts 17 may comprise a disposable plastic and/or metal material configured for providing a sterile surface within one of the one or more passages 15 in the freezable device 10.

FIGS. 6A-6B illustrate bottom views of various alternative freezable devices 20, 30, 40 according to various embodiments. Referring initially to FIG. 5A, the freezable device 20 may define a “Y” shape, such that the passage 25 is defined as a notch in a side of the freezable device 20. In such embodiments, the passage surface may be defined as a portion of the perimeter surface extending around the perimeter of the freezable device 20. Moreover, as discussed above in reference to freezable device 10, the passage 25 may extend through the entirety of the freezable device 20 through a first surface (not shown) and a second surface 23 opposite the first surface. Moreover, at least one of the first surface and the second surface 23 may have a plurality of protrusions 26 distributed thereon.

Referring now to FIG. 5B, a freezable device 30 may have a substantially rectangular shape, although the freezable device 30 may have a variety of other shapes (e.g., square, round, elliptical, and/or the like). As illustrated in FIG. 5B, the freezable device 30 may define a continuous shape, such that no passage extends therethrough. Moreover, as illustrated, at least one of a first surface (not shown) and a second surface 33 opposite the first surface may have a plurality of protrusions 36 distributed thereon.

In reference to FIG. 5C, a freezable device 40 may have a plurality of passages 45 extending therethrough. The freezable device 40 may define a plurality of protrusions 46 distributed around the plurality of passages 45 on the second surface 43. Particularly in configurations in which the freezable device 40 is sterile, the freezable device 40 may be used for multiple needle pricks (e.g., multiple injections and/or blood draws) from a patient, which utilizing a previously unused passage 45 for each needle prick. Thus, each needle may be inserted into the patient through an otherwise unused passage 45.

### First Exemplary Method of Use

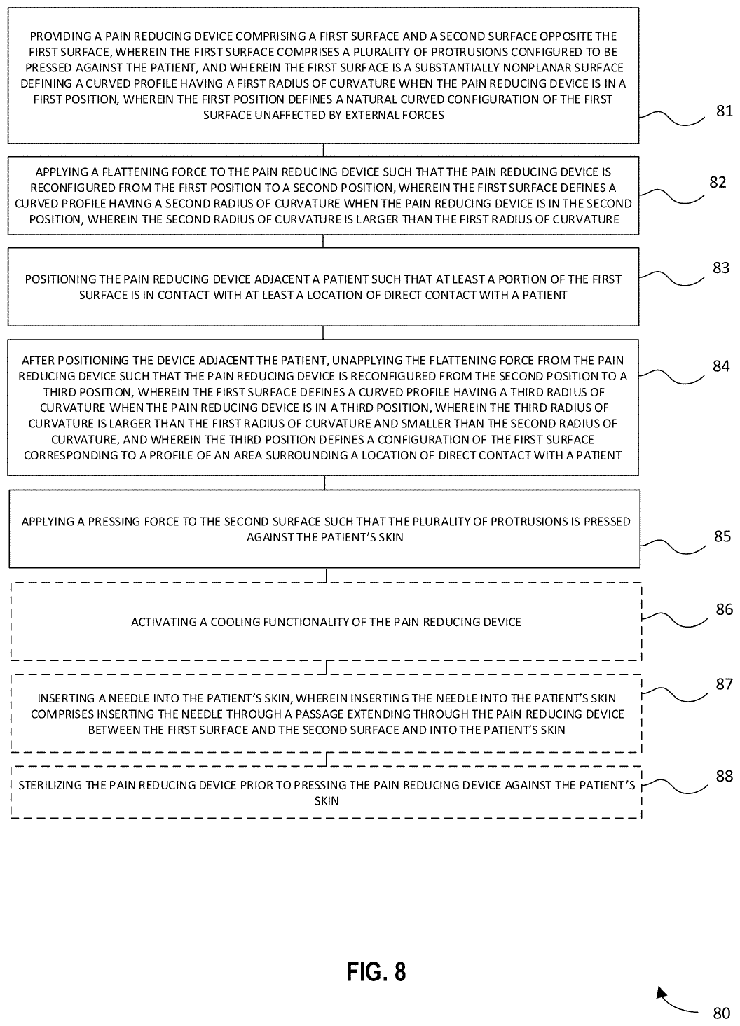

FIG. 8 illustrates a flow diagram of an exemplary method of reducing perceived pain during a needle insertion into a patient according to an exemplary embodiment described herein.

At block 81, a pain reducing device comprising a first surface and a second surface opposite the first surface may be provided. In various embodiments, the first surface may comprise a plurality of protrusions configured to be pressed against the patient. In various embodiments, the first surface may be a substantially nonplanar surface defining a curved profile having a first radius of curvature when the pain reducing device is in a first position. In various embodiments, the first position masy define a natural curved configuration of the first surface unaffected by external forces.

At block 82, a flattening force may be applied to the pain reducing device such that the pain reducing device is reconfigured from the first position to a second position. In various embodiments, the first surface may define a curved profile having a second radius of curvature when the pain reducing device is in the second position. The second radius of curvature may be larger than the first radius of curvature. In various embodiments, the second position may define a substantially flat configuration of the first surface affected by the flattening force.

At block 83, the pain reducing device may be positioned adjacent a patient such that at least a portion of the first surface is in contact with at least a location of direct contact with a patient.

At block 84, after positioning the device adjacent the patient, the flattening force may be unapplied from the pain reducing device such that the pain reducing device is reconfigured from the second position to a third position. In various embodiments, wherein the first surface may define a curved profile having a third radius of curvature when the pain reducing device is in a third position. The third radius of curvature may be larger than the first radius of curvature and smaller than the second radius of curvature. The third position may define a configuration of the first surface corresponding to a profile of an area surrounding a location of direct contact with a patient. In various embodiments, because the first position of the freezable device may represent the natural configuration of the device, and because the first surface of the freezable device may have a larger radius of curvature in the third position than in the first position, a pinching force may be applied to the patient body by the pain reducing device in the third position as a result of the patient body preventing the flexible shell comprised of pliable material from returning to the first position (i.e. its natural configuration).

At block 85, a pressing force may be applied to the second surface such that the plurality of protrusions is pressed against the patient's skin. In various embodiments, both a pressing force and a pinching force may be applied to the patient body. In various embodiments, a pressing force and a pinching force may be applied to the patient body in different directions relative to the surface of the patient body. In various embodiments, for example, the pressing force may be applied to the patient body in a direction perpendicular to the surface of the patient body, while the pinching force may be applied to the patient body in a different direction corresponding to the third radius of curvature to the surface of the patient body. In such a configuration, the pinching force and the pressing force may respectively provide different sensory confusion sensation to the patient, thereby providing an increased reduction in perceived pain caused by a needle insertion.

At block 86, a cooling functionality of the pain reducing device may be activated. In various embodiments, activating a cooling functionality of the pain reducing device may comprise breaking a barrier within an interior cavity of the pain reducing device so as to initiate an interaction between a first substance and a second substance positioned within the interior cavity, wherein the first substance and the second substance are separated by the barrier when the pain reducing device is in the first position. In various embodiments, the first substance may comprise water and the second substance may comprise ammonium chloride.

At block 87, a needle may be inserted into the patient's skin. In various embodiments, inserting the needle into the patient's skin may comprise inserting the needle through a passage extending through the pain reducing device between the first surface and the second surface and into the patient's skin. In various embodiments, the needle may be inserted into the patient's skin proximate the location of direct contact with a patient. In various embodiments, a membrane may cover at least one end of the passage along a perimeter surface of the pain reducing device and inserting the needle through the passage and into the patient's skin may further comprise puncturing the membrane.

At block 88, the pain reducing device may be sterilized prior to pressing the pain reducing device against the patient's skin. In various embodiments, for example, a plurality of sterile pain reducing devices may be stored within a dispenser in a freezer and may be removed as needed. In various embodiments, the pain reducing device may be configured to be used for a single needle insertion prior to disposal.

### Second Exemplary Method of Use

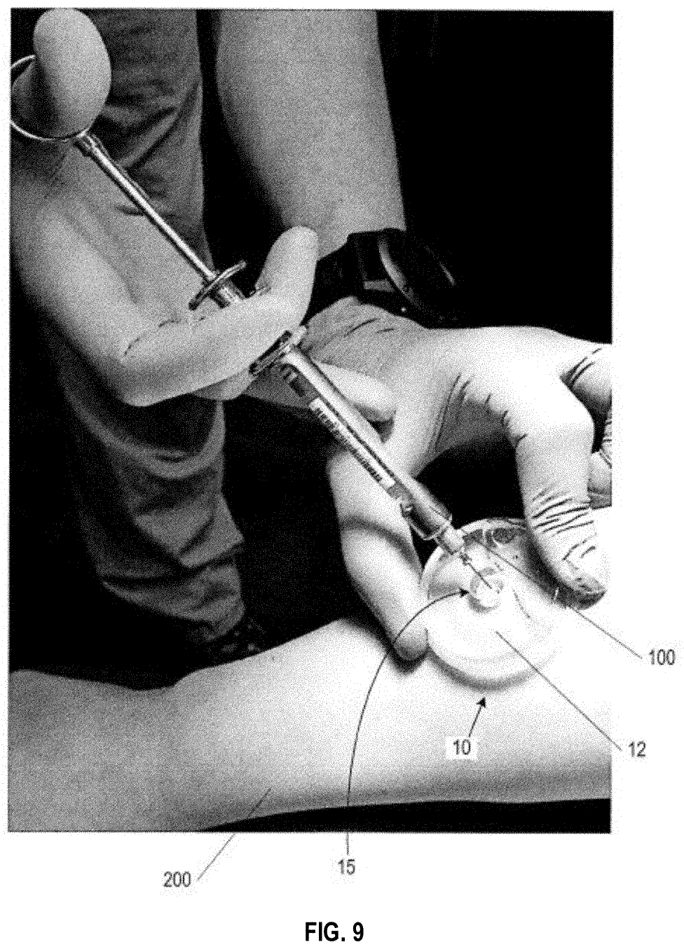

FIG. 9 illustrates a freezable device 10 in use. In various embodiments, the freezable device 10 may be cooled to freeze or otherwise chill the freezable substance or gel located within the interior cavity of the shell 11. For example, the freezable device 10 may be stored within a residential, commercial, and/or medical-grade freezer prior to use. After removal from the freezer, in various embodiments, the insert 17 may be inserted into the passage 15 of the freezable device 10.

The cooled freezable device 10 may be placed against the skin of a patient 200 such that at least a portion of the passage 15 is aligned with the needle insertion site at which a needle 100 is intended to be inserted. The cooled freezable device 10 is placed against the patient's skin such that at least some of the plurality of protrusions are in contact with the patient's skin. The freezable device 10 is depressed against the skin of the patient 200 such that the protrusions form corresponding indentions in the skin of the patient.

While the freezable device 10 is pressed against the skin of the patient 200, the needle 100 is inserted through the passage 15 and into the patient 200. In use, the freezable device 10 reduces perceived pain resulting from the needle insertion by providing a chilled surface at least partially around the needle insertion site, as well as providing a plurality of high pressure contact points by the plurality of protrusions being depressed into the surface of the patient's skin. The sensations perceived by the patients that are attributable to the cold-temperature of the freezable device 10 and the pressure points of the protrusions provide a localized sensory confusion sensation in the area surrounding the needle insertion site. Such sensory confusion may impede local pain receptors in the area immediately surrounding the needle insertion from providing a painful sensation felt by the patient.

### Conclusion

Many modifications and other embodiments of the inventions set forth herein will come to mind to one skilled in the art to which these inventions pertain having the benefit of the teachings presented in the foregoing descriptions and the following claims. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

For example, various embodiments of freezable device 10 may be sterile and provided for use by medical professionals. Such sterilized freezable device 10 may be disposable and intended for a single use prior to disposal. Alternatively, the freezable device 10 may be reusable and intended for residential use. As non-limiting examples, such a reusable freezable device 10 may be useful in providing insulin shots, injectable pain medication, and/or for other home-administered injections.

That which is claimed:

1. A pain reducing device comprising:
a flexible shell comprising a first non-planar surface and a second surface opposite the first surface; and
an interior cavity defined by the flexible shell and located intermediate the first surface and the second surface of the flexible shell,
wherein:
the flexible shell comprises a pliable material;
the first surface comprises a plurality of protrusions; and
the interior cavity is at least partially filled with a first substance.

2. The pain reducing device of claim 1, wherein the first non-planar surface defines a curved profile having a first radius of curvature when the pain reducing device is in a first position, the first position being a natural curved configuration with the first surface unaffected by any externally applied forces.

3. The pain reducing device of claim 2, wherein the first surface defines a curved profile having a second radius of curvature when the pain reducing device is in a second position, wherein the second radius of curvature is larger than the first radius of curvature, and wherein the second position defines a substantially flat configuration of the first surface affected by an applied force.

4. The pain reducing device of claim 3, wherein the first surface defines a curved profile having a third radius of curvature when the pain reducing device is in a third position, wherein the third radius of curvature is larger than the first radius of curvature and smaller than the second radius of curvature, and wherein the third position defines a configuration of the first surface corresponding to a profile of an area surrounding a location of direct contact with a patient.

5. The pain reducing device of claim 4, wherein the second surface is a nonplanar surface defining a curved profile that corresponds to the curved profile of the first surface in the first position.

6. The pain reducing device of claim 1, further comprising at least one passage extending through the device from the first surface to the second surface, wherein a passage is configured to surround at least a portion of a location of direct contact with a patient

7. The pain reducing device of claim 6, wherein the flexible shell comprises a perimeter surface surrounding the shell and extending between the first surface and the second surface to define the interior cavity of the shell; and
wherein the passage defines a passage surface at least partially surrounding the passage and extending between the first surface and the second surface; and
wherein the perimeter surface comprises the passage surface.

8. The pain reducing device of claim 6, wherein the passage defines a first hole extending through the first surface and a second hole extending through the second surface, and a passage perimeter surface extending between the first hole and the second hole.

9. The pain reducing device of claim 6, comprising two or more passages extending through the device from the first surface to the second surface.

10. The pain reducing device of claim 1, wherein the flexible shell is sterile.

11. The pain reducing device of claim 1, wherein the first substance is a gel.

12. The pain reducing device of claim 1, further comprising a second substance within the interior cavity of the flexible shell, wherein the interior cavity defines a first portion and a second portion separated by a barrier when the flexible device is in the first position, and wherein the first substance and the second substance are positioned within the first portion and the second portion, respectively.

13. The pain reducing device of claim 12, wherein the barrier is configured to break when the pain reducing device is moved from a first position to a second position so as to initiate an interaction between the first substance and the second substance, wherein the first substance and the second substance are configured such that the interaction activates a cooling functionality of the pain reducing device.

14. The pain reducing device of claim 13, wherein the first substance is water and the second substance is ammonium chloride.

15. A method of reducing perceived pain during a needle insertion into a patient, the method comprising for the steps of:
providing a pain reducing device comprising a first surface and a second surface opposite the first surface, wherein the first surface comprises a plurality of protrusions configured to be pressed against the patient, and wherein the first surface is a substantially nonplanar surface defining a curved profile having a first radius of curvature when the pain reducing device is in a first position, wherein the first position defines a natural curved configuration of the first surface unaffected by external forces;
applying a flattening force to the pain reducing device such that the pain reducing device is reconfigured from the first position to a second position, wherein the first surface defines a curved profile having a second radius of curvature when the pain reducing device is in the second position, wherein the second radius of curvature is larger than the first radius of curvature;
positioning the pain reducing device adjacent a patient such that at least a portion of the first surface is in contact with at least a location of direct contact with a patient;
after positioning the device adjacent the patient, unapplying the flattening force from the pain reducing device such that the pain reducing device is reconfigured from the second position to a third position, wherein the first surface defines a curved profile having a third radius of curvature when the pain reducing device is in a third position, wherein the third radius of curvature is larger than the first radius of curvature and smaller than the second radius of curvature, and wherein the third position defines a configuration of the first surface corresponding to a profile of an area surrounding a location of direct contact with a patient;
applying a pressing force to the second surface such that the plurality of protrusions is pressed against the patient's skin.

16. The method of claim 15, wherein the second position defines a substantially flat configuration of the first surface affected by the flattening force.

17. The method of claim 15, further comprising activating a cooling functionality of the pain reducing device.

18. The method of claim 17, wherein activating a cooling functionality of the pain reducing device comprises breaking a barrier within an interior cavity of the pain reducing device so as to initiate an interaction between a first substance and a second substance positioned within the interior cavity, wherein the first substance and the second substance are separated by the barrier when the pain reducing device is in the first position.

19. The method of claim 15, further comprising inserting a needle into the patient's skin, wherein inserting the needle into the patient's skin comprises inserting the needle through a passage extending through the pain reducing device between the first surface and the second surface and into the patient's skin.

20. The method of claim 19, wherein the needle is inserted into the patient's skin proximate the location of direct contact with a patient.

21. The method of claim 19, wherein a membrane covers at least one end of the passage along a perimeter surface of the pain reducing device, and wherein inserting the needle through the passage and into the patient's skin further comprises puncturing the membrane.

22. The method of claim 15, further comprising steps for sterilizing the pain reducing device prior to pressing the pain reducing device against the patient's skin.
